(19) United States
(12) Patent Application Publication
(10) Pub. No.: US 2014/0250574 A1
(43) Pub. Date: Sep. 11, 2014

(54) ADJUSTING STRAP, AND SWIMMING GOGGLES AND SWIM FINS COMPRISING THE SAME

(71) Applicant: QBAS CO., LTD., Taipei (TW)

(72) Inventor: Chih-Cheng SHIUE, Taipei (TW)

(73) Assignee: QBAS CO., LTD., Taipei (TW)

(21) Appl. No.: 14/200,959

(22) Filed: Mar. 7, 2014

(30) Foreign Application Priority Data
Mar. 8, 2013 (TW) 102204387
May 2, 2013 (TW) 102208137

(51) Int. Cl. A44B 1/08; A63B 31/08; A44B 11/22; A61F 9/02

(52) U.S. Cl. CPC A44B 1/08; A61F 9/027; A63B 31/08; A44B 11/22
USPC 2/452; 24/101 R; 441/55

(57) ABSTRACT

An adjusting strap is provided. The adjusting strap has a strap body and at least one fastening portion. An end zone of the strap body has at least one fastening hole, which is disposed along the strap body in a lengthwise direction. The fastening portion is disposed on the strap body and located at an inside area adjacent to the fastening hole, the at least one fastening portion is integrally formed with the strap body and extends away from a top surface of the strap body. When the end zone of the strap is bent in reverse, the fastening portion is capable of threading through the fastening hole to be in position.

This application claims the benefits of the priorities based on Taiwan Utility Model Application No. 102204387 filed on Mar. 8, 2013 and No. 102208137 filed on May 2, 2013, the disclosures of which are incorporated herein by reference in their entirety.

### CROSS-REFERENCES TO RELATED APPLICATIONS

Not applicable.

### BACKGROUND OF THE INVENTION

1. Field of the Invention

The present invention provides an adjusting strap, a goggle and a swimming fin comprising the adjusting strap. The adjusting strap is capable of conveniently adjusting the tension with positioning and fixing functions.

2. Descriptions of the Related Art

Underwater devices, such as swimming goggles, diving goggles and swimming fins, are used by those participating in water sports. When such an underwater device is worn by the user, usually a set of fixing structures on the device is used to adjust the tension level of the underwater device so that the underwater device is tied to a part of the body.

However, the conventional adjusting and fixing approach is not comfortable or convenient for the user. In detail, as shown in FIG. 1, an adjusting structure 1 of the conventional swimming goggles comprises an elastic strap 11 and a connector 12 used in combination with the elastic strap 11. Because the connector 12 has a rod (not shown) therein, the elastic strap 11 is adapted to wrap around the rod in the connector 12. The user then adjusts the length of the elastic strap 11 that passes through the connector 12 according to his or her head size. An exposed end portion 111 of the elastic strap 11 is fixed by means of an annular piece 13 disposed on the elastic strap 11.

However, conventional adjusting structures 11 need multiple elements, so the production cost is high. Additionally, when the conventional adjusting structures is worn by the user, the user's skin will be pressed by the annular piece 13. The end portion 111 in excess tends to be detached from the annular piece 13 during the sport. Therefore, apart from being uncomfortable to use, the conventional adjusting structure 11 tends to be released unexpectedly.

In view of the shortcomings, it is important to provide an adjusting strap, suitable for different devices, that is adjustable in real time, convenient and comfortable to use.

### SUMMARY OF THE INVENTION

To solve the problems with the aforesaid fixing structure, an objective of the present invention is to provide an adjusting strap which is applicable to devices, such as water goggles, swimming goggles or swimming fins. With the special structure of the adjusting strap, the length of the strap body can be adjusted easily and rapidly. The position of the strap body can be fixed so that the adjusting strap can be worn with the most comfortable size by the user.

A first embodiment of the present invention provides an adjusting strap, which comprises a strap body and at least one fastening portion. The strap body has at least one fastening hole in an end zone of the strap body. The fastening hole is disposed along the strap body in a lengthwise direction. The fastening portion is disposed on the strap body and located at an inside area adjacent to the fastening hole, and the at least one fastening portion is integrally formed with the strap body and extends away from a top surface of the strap body. The fastening portion is able to be threaded through the fastening hole to be in position when the end zone of the strap body is bent in reverse.

A second embodiment of the present invention provides a swimming goggle, which comprises a goggle body and the adjusting strap that is described in the first embodiment. An side end of the goggle body is provided with a fastening loop for the end zone of the adjusting strap to pass therethrough and to be bent in reverse.

A third embodiment of the present invention provides a swimming fin, which comprises a fin body and the adjusting strap described in the first embodiment. An side of the fin body is provided with a fastening loop for the end zone of the adjusting strap to pass therethrough and to be bent in reverse.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 is a schematic view of an adjusting structure of a conventional pair of swimming goggles;

FIG. 2 is a top view of an adjusting strap according to the first embodiment of the present invention;

FIG. 3A is a partial perspective view of the adjusting strap according to the first embodiment of the present invention;

FIG. 3B is a partial perspective view of the first embodiment of the present invention in a usage state;

FIG. 4 is a partial top view of the adjusting strap according to the first embodiment of the present invention;

FIG. 5 is a partial side view of a fastening portion according to the first embodiment of the present invention; and

FIG. 6 is a partial schematic view of a pair of swimming goggles according to the second embodiment of the present invention in a usage state.

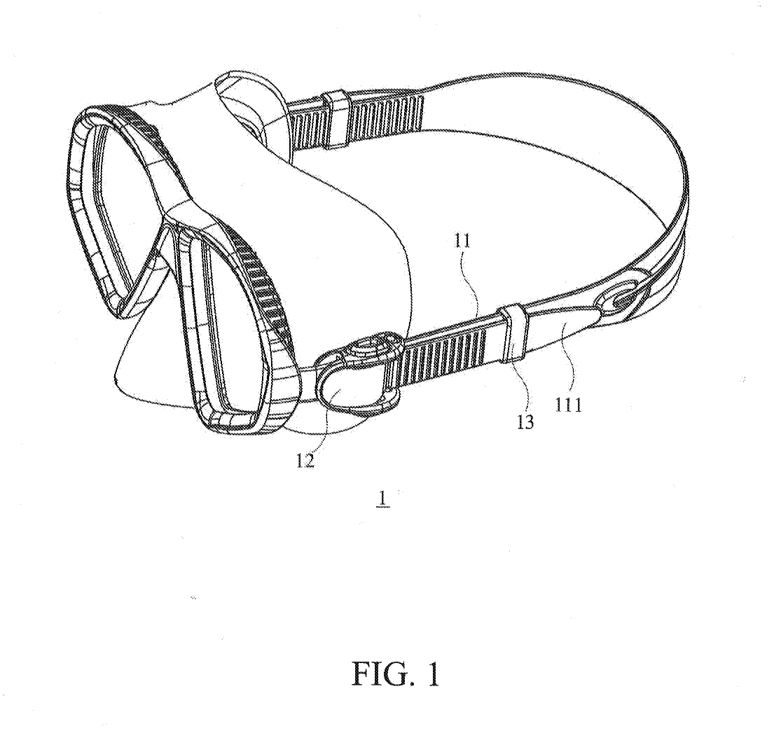

### DESCRIPTION OF THE PREFERRED EMBODIMENT

In the following description, the present invention will be explained with reference to embodiments thereof. However, the description of these embodiments is only intended to illustrate the technical contents of the present invention as well as objectives and effects thereof, but not to limit the present invention. It should be appreciated that in the following embodiments and attached drawings, elements unrelated to the present invention are omitted from depiction. The dimensional and relative positional relationships among individual elements in the attached drawings are illustrated only for ease of understanding, but not to limit the actual scale and sizes.

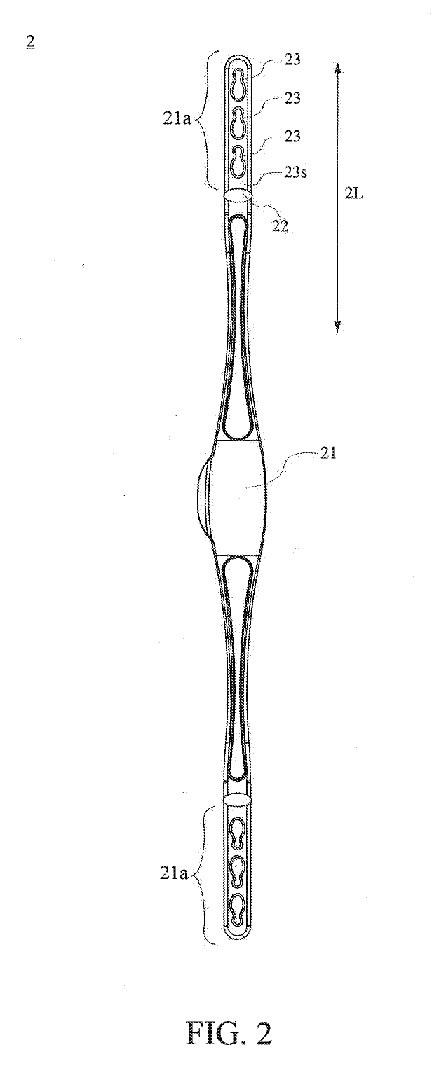

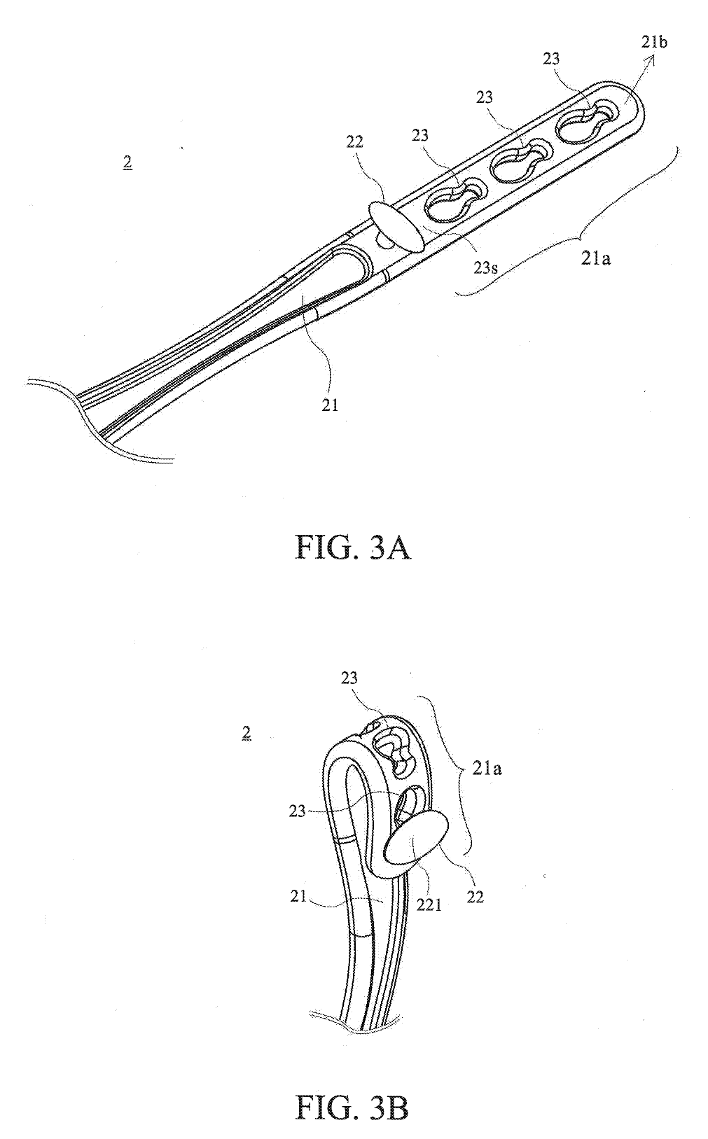

For the first embodiment of the present invention, structural features of individual parts will be described with reference to FIGS. 2, 3A, 4 and 5. The usage state of the adjusting strap will be described with reference to FIG. 3B.

FIG. 2 illustrates the top view of an adjusting strap 2 according to the first embodiment in a flatly stretched state. In this embodiment, the adjusting strap 2 comprises a strap body 21 and at least one fastening portion 22. An end zone 21a is defined at each of the two opposite ends of the strap body 21 respectively. At least one fastening hole 23 is formed in each end zone 21a of the strap body 21. The fastening hole 23 is disposed in the lengthwise direction 2L of the adjusting strap 2.

Specifically, as shown in FIG. 2, the two opposite ends of the strap body 21 are each formed with a fastening portion 22 that is adapted to mate with the fastening hole 23 of the end zone 21a. The number of fastening portions is not limited to what is illustrated in this embodiment; and instead, one or more fastening portions may be additionally disposed depending on practical needs.

It shall be appreciated that the number of fastening holes may be decided according to the length of each end zone of the strap body as can be devised by those skilled in the art. In detail, to obtain a wide range in which tension of the adjusting strap can be selected, two or more fastening holes 23 with an identical size may be formed according to the size of the upper end zone. The fastening holes 23 are arranged equidistantly. Therefore, FIGS. 2, 3A, 4 and 5 only illustrate the arrangement of the fastening holes 23, but are not intended to limit the number of the fastening holes 23 in actual implementations.

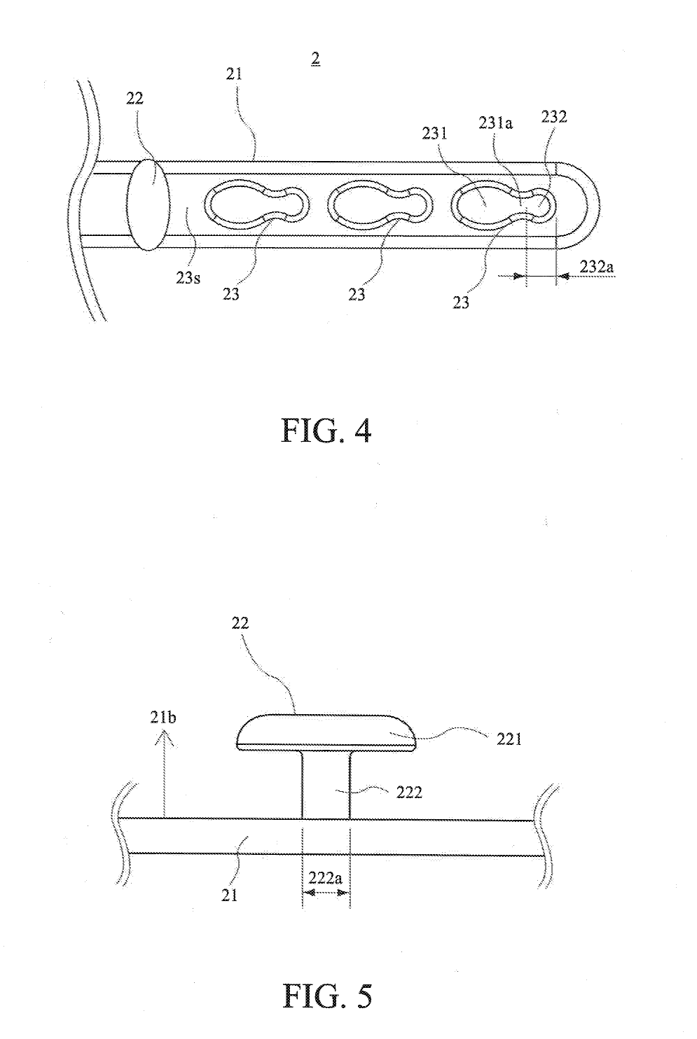

With reference to both FIGS. 3A and 4, a partial perspective view and a partial top view of the adjusting strap 2 according to the first embodiment are shown therein respectively. The fastening portion 22 is disposed on the strap body and located at an inside 23s area adjacent to the fastening holes 23 and the fastening portion is integrally formed with the strap body and extends away from a top surface 21b of the strap body 21 (as shown in FIG. 5). In this embodiment, the strap body 21 is preferably formed of an elastic rubber. The fastening portion 22 is formed of a material that is harder than the strap body 21. As can be devised by those skilled in the art, the strap body and the fastening portion that are made of different materials may be integrally formed through over-molding into a unitary member. Alternatively, to simplify the manufacturing process and save the use of other materials, those skilled in the art may also devise to form the fastening portion and the strap body of a same elastic rubber material so that the additional over-molding process can be eliminated so that the fastening portion and the strap body can be formed into a unitary member through a single manufacturing process.

As shown in FIG. 4, each of the fastening holes 23 has a large aperture portion 231 and a small aperture portion 232 communicating with each other. The small aperture portion 231 is disposed on an outer side of the large aperture portion 231 communicating therewith.

FIG. 5 illustrates a partial side view of a fastening portion 22 according to the first embodiment. The fastening portion 22 comprises a head portion 221 and a pillar portion 222 connected between the head portion 221 and the strap body 21. The head portion 221 has an overall size greater than the pillar portion 222.

Thus, the structure of the adjusting structure of the first embodiment has been described above. Hereinbelow, the method of using the adjusting strap 2 of the first embodiment will be described.

As will be appreciated by those skilled in the art, the strap body 21 is bendable because it is formed from an elastic rubber material. With reference to both FIGS. 3B and 4, when the end zone 21a of the strap body 21 is bent in reverse towards the fastening portion 22, the head portion 221 of the fastening portion 22 is capable of threading through the elastic large aperture portion 231 of the fastening hole 23 easily to position the pillar portion 222 into the small aperture portion 232 communicating with the large aperture portion 231. Then, when the strap body 21 is resiliently forced, the fastening portion 22 moves in a direction away from the large aperture portion 231 and is reliably received in the small aperture portion 232.

In detail, when the length of the adjusting strap is to be adjusted, the user can bend the end zone 21a of the strap body 21 in reverse and choose one of the fastening holes 23 to thread the head portion 221 of the fastening portion 22 through the elastic large aperture portion 232 of the chosen fastening hole 23. To prevent disengagement of the fastening portion 22 from the fastening hole 23, a force needs to be applied to push the pillar portion 222 of the fastening portion 22 towards the elastic small aperture portion 232 so that the fastening portion 22 is reliably fixed in the small aperture portion 232.

It shall be appreciated that, as shown in FIG. 4 and FIG. 5, an outer diameter 222a of the pillar portion 222 is substantially equal to or slightly greater than an inner diameter 232a of the small aperture portion 232. Thus, the pillar portion 222 of the fastening portion 22 can be positioned in the small aperture portion 232 securely without the possibility of sliding back into the large aperture portion 231. From the above description, those skilled in the art can devise the following way of releasing and detaching the fastening portion 22 from the fastening hole 23: simply by applying a force in a reverse direction to push the pillar portion 222 of the fastening portion 22 back to the large aperture portion 231. The head portion 221 of the fastening portion 22 can be detached from the strap body 21 via the large aperture portion 231. Then, the end zone 21a of the strap body 21 will restore the state as it was prior to the reverse bending.

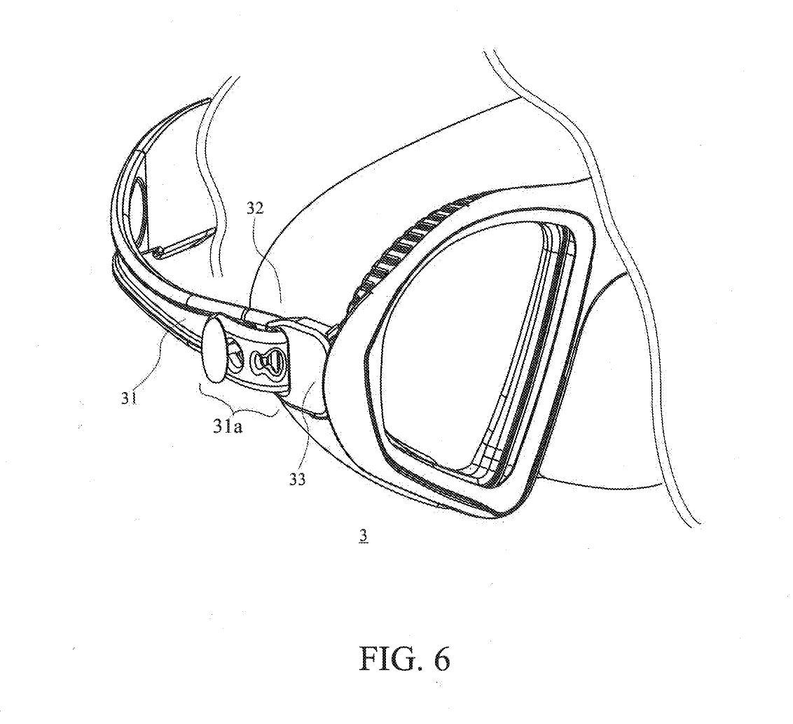

The second embodiment of the present invention provides a swimming goggle that is worn by means of the adjusting strap of the first embodiment. As shown in FIG. 6, a partial perspective view of the swimming goggle of the second embodiment is shown therein. Because the technical features of the adjusting strap 31 of the second embodiment make no difference from those of the first embodiment, the structure and operations of the adjusting strap 3 will not be described again herein. However, it shall be particularly appreciated that the second embodiment differs from the first embodiment mainly in that a side end 32 of the goggle body 3 is provided with a fastening loop 33 for the end zone 31a of the adjusting strap 31 to pass therethrough and to be bent in reverse.

A third embodiment of the present invention provides a swimming fin (not shown) that is worn by means of the adjusting strap of the first embodiment. Because the technical features of the adjusting strap of the third embodiment make no difference from those of the first embodiment, the structure and operations of the adjusting strap in the third embodiment will not be described again herein. However, it shall be particularly appreciated that the third embodiment differs from the first embodiment mainly in that a side of the fin body is provided with a fastening loop for the end zone of the adjusting strap to pass therethrough and to be bent in reverse. From the above description of the swimming goggle, those skilled in the art can readily devise the position of the fastening loop of the swimming fin so the swimming fin will not be additionally depicted.

According to the above descriptions, through the simple use of the fastening portion and the fastening holes in combination on the adjusting strap, the present invention can reduce the number of additional fixing members disposed in the swimming goggle and the swimming fins to lower the manufacturing cost. The user wearing the swimming goggle or the swimming fin can position and fix the swimming goggle or the swimming fin by operating the adjusting strap directly. No additional part on the strap body comes into contact with the user's skin to cause discomfort. Thereby, the adjusting strap of the present invention has advantages of being convenient and comfortable to use with rapid adjustment.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. An adjusting strap, comprising:
a strap body, having at least one fastening hole in an end zone of the strap body, the fastening hole being disposed along the strap body in a lengthwise direction;
at least one fastening portion, being disposed on the strap body and located at an inside area adjacent to the fastening hole, the at least one fastening portion being integrally formed with the strap body and extending away from a top surface of the strap body, wherein the fastening portion is capable of threading through the fastening hole to be in position when the end zone of the strap body is bent in reverse.

2. An adjusting strap as claimed in claim 1, wherein the fastening hole has a large aperture portion and a small aperture portion communicating with each other, and the small aperture portion is disposed at an outer side of the large aperture portion communicating therewith.

3. An adjusting strap as claimed in claim 2, wherein the fastening portion comprises a head portion and a pillar portion connected between the head portion and the strap body, the head portion is capable of threading through the large aperture portion of the fastening hole so that the pillar portion is positioned in the small aperture portion communicating with the large aperture portion when the end zone of the strap body is bent in reverse.

4. An adjusting strap as claimed in claim 2, wherein the strap body and the fastening portion are formed of an elastic rubber, wherein the fastening portion moves in a direction away from the large aperture portion so as to stably contain the fastening portion in the small aperture portion when the strap body is resiliently forced.

5. An adjusting strap as claimed in claim 4, wherein the material of the fastening portion is harder than the strap body.

6. An adjusting strap as claimed in claim 3, wherein an outer diameter of the pillar portion is substantially equal to or slightly greater than an inner diameter of the small aperture portion.

7. An adjusting strap as claimed in claim 1, further comprising a plurality of fastening holes arranged equidistantly in the end zone of the strap body.

8. A swimming goggle, comprising a goggle body and the adjusting strap as claimed in claim 1, wherein a side end of the goggle body is provided with a fastening loop for the end zone of the adjusting strap to pass therethrough and to be bent in reverse.

9. A swimming fin, comprising a fin body and the adjusting strap as claimed in claim 1, wherein a side of the fin body is provided with a fastening loop for the end zone of the adjusting strap to pass therethrough and to be bent in reverse.
